(19) United States
(12) Patent Application Publication
(10) Pub. No.: US 2010/0268040 A1
(43) Pub. Date: Oct. 21, 2010

(54) METHOD AND SYSTEM FOR DETECTION OF PRE-FAINTING AND OTHER CONDITIONS HAZARDOUS TO THE HEALTH OF A PATIENT

(76) Inventors: Ilan Ben-Oren, Modi'in (IL); Yaron Ilan, Jerusalem (IL)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/747,418

(22) PCT Filed: Dec. 10, 2008
(86) PCT No.: PCT/IL08/01600

§ 371 Date: Jun. 10, 2010

(30) Foreign Application Priority Data
Dec. 10, 2007 (IL) 188033

(51) Int. Cl. A61B 5/00

(52) U.S. Cl. 600/301

(57) ABSTRACT

Included herein are embodiments relating to detecting and warning of the presence of pre-fainting and other conditions that may be hazardous to the health of a patient having one or more types of disease or disorder. A wide range of physiological and physical parameters can be monitored and logically and/or mathematically related to determine the value of a new parameter, the risk parameter alpha. The parameters that appear in the function can be selected according to the patient's known pathological condition. An initial threshold value for alpha can be determined and compared with a current value of alpha. If the comparison shows that there exists danger of the onset of a pre-fainting and/or other medically hazardous condition, a warning signal can be emitted. The current value of alpha can be continually determined and, if relevant, updated according to the history of the patient.

### FIELD OF THE INVENTION

The present invention relates to the field of medical care and diagnostics. Specifically, this invention relates to a system and method for detecting pre-loss of consciousness, pre-syncope, or a syncope or other conditions that are risky/hazardous to a patient.

### BACKGROUND OF THE INVENTION

As is well known, many medical conditions, including cardiovascular diseases, diabetes, disorders causing apnea, neurological disorders, etc., may result in loss of consciousness, pre syncope and syncope, and other similar conditions and to conditions in which the patient cannot respond, or the condition is asymptotic e.g., pre-stroke, epileptic seizures, or pregnancy related conditions. As a matter of convenience, the term “pre-fainting” will be defined herein to include any condition in which the afflicted patient does not receive, does not properly interpret or is unable to respond to early warning signs of an impending medical problem.

Syncope is the mechanism by which cardiovascular abnormalities may cause falls in older people. Syncope is a symptom, defined as a transient, self-limited loss of consciousness, usually leading to falling. The onset of syncope is relatively rapid, and the subsequent recovery is spontaneous, complete and usually prompt. Irrespective of the precise cause underlying a syncopal event, a sudden cessation of cerebral blood flow for 6-8 seconds and/or a decrease in systolic blood pressure to 60 mm Hg has been shown to be sufficient to cause complete loss of consciousness. Further, it has been estimated that as small as a 20% drop in cerebral oxygen delivery is sufficient to cause loss of consciousness. Age-associated physiological changes in heart rate, blood pressure, cerebral blood flow, baroreflex sensitivity and intravascular volume regulation, combined with comorbid conditions and concurrent medications, may all contribute to the higher incidence of syncope in the older population. In terms of the immediate injurious consequences of syncope, major morbidities such as fractures and motor vehicle accidents have been reported in 6% of patients and minor injury such as laceration and bruises in 29%. Recurrent syncope is associated with fractures and soft-tissue injury in 12% of patients.

Syncope must be differentiated from other ‘non-syncopal’ conditions associated with real or apparent transient loss of consciousness. This differentiation is less difficult in the situation where falls without loss of consciousness are the presenting problem. However, Differentiating syncope from other causes of falls is sometimes a difficult task especially in advanced age, and up to one-quarter of syncopal events will present as unexplained falls. The following are some critical issues that contribute to uncertainty in the diagnostic evaluation:
Amnesia for loss of consciousness makes the acquisition of an accurate history difficult.
Cognitive impairment influences the accuracy of recall for events.
Gait and balance instability and slow protective reflexes are frequent in community-dwelling older people; in these circumstances moderate haemodynamic changes insufficient to cause syncope may result in falls.

It is important, therefore, to make every attempt to obtain a witness account of episodes, although this is not available in many instances.

Falls occur commonly in older persons and are the seventh leading cause of death. Falls are associated with functional deterioration and “fear of falling”.

Patients are in particular danger of loosing consciousness prior to the diagnosis of their illnesses though they remain in danger also once their disease is chronic and at such times may loose consciousness with no apparent warning signs whatsoever. For example, many patients, including cardiac patients and diabetics, report that at times, the symptoms of their disease come on rapidly, and they are unable to react in time to prevent loss of consciousness. Furthermore, apnea and other disorders may occur during sleeping periods, thereby causing loss of consciousness, which may eventually lead to injury, accident or even death, depending on the basic disease.

In the past, many patients were confined to hospitals for long periods of time in order to enable the medical staff to monitor their vital signs thereby saving the lives of many of them. However, hospital confinement is extremely inconvenient for patients, and further, exposes the patients to various types of contagious diseases. In addition, the hospitalization itself is expensive, and further, causes children to stay out of school, and adults to miss work, thereby causing both social and economic difficulties. As loss of consciousness is common and costly especially in the elderly, presentation and prevalence may be different compared with the young.

Moreover, quality of life may decrease for at least one year after each loss of consciousness episode, especially in patients who are older, have recurrent episodes, a neurological or psychogenic diagnosis, and a higher level of co-morbidity.

In patients one year after syncope, four independent predictors of serious arrhythmia or death were identified, including abnormal EEG, age older than 45 years, history of congestive heart failure and history of ventricular arrhythmia. The risk of death in the year following the episode ranges from 1% in patients with no risk factors to 27% in patients with three or more risk factors. In addition, within 30 days of syncope, five risk factors were identified in patients leading to serious outcomes (e.g., death, myocardial infarction, significant hemorrhage, pulmonary embolism, arrhythmia, stroke), which include systolic blood pressure less than 90 mm Hg, shortness of breath, nonsinus rhythm or new changes present on ECG, history of congestive heart failure, and a hematocrit level less than 30 percent. Patients with any one risk factor had a 15.2 percent risk of serious outcome compared with a 0.3 percent risk for patients with no risk factors.

Sudden cardiac death is the most devastating complication of hypertrophic cardiomyopathy (HCM). Since HCM may present at young age, and since the risk period for sudden arrhythmic death may be long, decision-making in HCM patients may be difficult, and have lifelong implications.

Despite the fact that the patient might be unaware that he is about to undergo an episode of loss of consciousness, the transition to such a condition may in many cases preceded by changes in physical parameters such as temperature or blood pressure or by changes in body chemistry, such as glucose level in the blood.

In light of the above, many portable monitors have been developed wherein each monitor specifically detects parameter/s relevant to a specific medical condition, e.g., the measurement of glucose levels in diabetic patients, thereby warning either the patient or sending signals to a medical device attached to him or a remote medical analysis facility of any deviations from normal in the patient's condition.

U.S. Pat. No. 6,893,401, for example, relates to pulse transition time, therefore monitoring blood pressure at two different points on the patients body. The invention of U.S. Pat. No. 6,893,401 aims mainly at cardiovascular patients, and monitors a sole parameter, i.e. blood pressure.

U.S. Pat. No. 6,102,856 relates to a wearable vital sign monitor designed for cardiovascular disturbances. Accordingly, the parameters measured in U.S. Pat. No. 6,102,856 are all related to cardiovascular diseases, including ECG data, respiration rate, pulse rate, etc. Although the method of U.S. Pat. No. 6,102,856 relates to the measurement of a number of parameters they are all connected to cardiovascular disturbances, therefore, only such patients may benefit form the vital sign monitor of U.S. Pat. No. 6,102,856.

As this brief review of the prior art reveals many non-invasive monitors have been developed, each monitor is directed to a specific parameter or a group of parameters. These monitors are generic in the sense that they are designed to measure specific parameters in contrast to detecting a specific condition. Once a value of a parameter that falls outside of a range of values that has been defined on a statistical basis to be normal the monitor might be activated to issue a warning, initiating administration of a drug, etc. There is no attempt in prior art to integrate the sensors in a way that would tailor the monitor to the particular needs of a specific individual based on his health history and in particular to learn from past experience the exact values of a specific measured parameter or group of parameters that are indicative, for that individual, of the onset of a loss of consciousness state as defined herein. Such individualization is highly desirable for many reasons including the fact that, despite the availability of voluminous statistical data related to different conditions, the definition of “normal” depends on many factors. For example, a specific level of glucose in the blood of one person might be easily tolerated and poses no potential threat, while for another person such might be indicative of hypoglycemia, and therefore indicates an impending loss of consciousness. Furthermore, for a particular individual who also suffers from chronic high blood pressure, the glucose level that is indicative of hypoglycemia might be significantly different from that of a person not suffering from high blood pressure.

It would therefore be highly desirable to develop devices and methods that can monitor individual patients in respect of a wide variety of parameters, wherein those parameters are related to the medical history of the patient and can relate to any type of disease or disorder which could cause loss of consciousness or pre-syncope and syncope or other conditions hazardous to the patient's health. The device should be self-learning and able to adjust the values at which it initiates an alarm or other action based on previous occurrence/s of the monitored phenomenon for the same patient. Such a system/method would significantly reduce the number of false alarms and decrease incidences of missed alarms. If an occurrence is missed, the system should be able to retrospectively identify the pattern associated with the condition and adjust the functions and/or thresholds used to determine if an alarm should be initiated accordingly. Thus, also missed alarms will contribute to the self learning of the device. In addition, the system/method should ensure that the patient, or any other appropriate party, be alerted to any abnormalities, thereby aiding in the early detection and treatment of conditions which may lead to loss of consciousness, and later on even to death.

It is an object of the present invention to provide a method and devices for monitoring patients at risk of, or suffering from, any type of disease or disorder that could lead to loss of consciousness and/or any other medically hazardous condition, the device/method issuing alarms or initiating other appropriate action, based on self-learning of the health history of the patient, when a pre-loss of consciousness condition is detected.

Further purposes and advantages of this invention will become apparent as the description proceeds.

### SUMMARY OF THE INVENTION

In a first aspect the invention is a method for the detection, qualitative evaluation, and warning of the presence of pre-fainting and other conditions that are hazardous to the health of a patient having one or more types of disease/disorder. The method of the invention comprises the following steps:
a. monitoring at least one physiological parameter selected according to the patient's known pathological condition;
b. determining the instantaneous value of the risk parameter (alpha(t));
c. assigning to alpha(t) at least one threshold value (alpha) whose value is determined based on known normal values as determined by statistical studies;
d. comparing the value of alpha(t) to the current value of alpha;
e. emitting a warning signal if the comparison shows that the value of alpha(t) is different from the value of alpha by an amount that exceeds a value predetermined for the patient;
f. using the instantaneous monitored values of the parameter to update alpha(t); and
g. repeating steps d to f.

Embodiments of the method comprise the additional step of re-determining and, if relevant, updating the current value of alpha according to the history of the patient between steps e and f.

In embodiments of the method, emitting a warning signal comprises presenting the probability that a pre-fainting or other condition that is hazardous to the health of the patient is occurring or will occur.

In embodiments of the invention, self-learning techniques are used to assist in continually updating the value of alpha and/or a function used to determine the value of alpha(t).

In preferred embodiments of the method of the invention at least one additional physiological or physical parameter, which is selected according to the patient's known pathological condition is monitored. In these embodiments the instantaneous value of the risk parameter (alpha(t) is determined from a function that combines the measured values of the one selected physiological parameter and of the at least one additional parameter and the threshold value (alpha) is determined by statistical studies. Combination of the measured values of the parameters can be done either mathematically or logically. The threshold value (alpha) can be determined either by combining the known normal values of the selected parameter and the known normal values the at least one additional parameter or by using normal values of the combination of the selected parameter and the at least one additional parameter.

In the preferred embodiments of the invention the method n self-learning techniques are used to assist in continually updating one or both of the function used to determine the value of alpha(t) and the threshold value (alpha).

In an embodiment of the basic embodiment of the invention instead of emitting a warning signal if a comparison shows that the value of alpha(t) is different from the value of alpha by an amount that exceeds a value predetermined for the patient, then a new parameter is selected and the steps of the method are carried out using the new parameter. In an embodiment of the preferred embodiments of the invention, instead of emitting a warning signal, a new set of parameters comprising additional or different parameters is selected and the steps of the method are carried out using the new set of parameters.

The physiological parameters monitored can be selected from the following: heart rate; low frequency modulation of pulse; oxygen saturation; breath rate; heart rhythm, including the detection of atrial and ventricular arrhythmias, any premature beats, or nodal rhythm; body temperature; blood sugar; quantities of any electrolyte; blood acid base balance; PCO₂ levels; blood pressure; blood flow; tissue conductivity; SPO₂; degree of sweating; blood flow in small vessels; Pulse Transit Time; ECG; impedance plethysmography; acoustic breath detection; drug levels; acid-base balance in the serum, and EtCO₂. The physical parameters can be selected from the following: number of steps taken, steps rate, and an indication of physical movement of the body as a whole or of parts of the body.

In a second aspect the invention is A system for carrying out the first aspect of the invention. The system comprises a processor; at least one sensor to measure the appropriate physiological and physical parameters; and a power supply.

The system according of the invention may additionally comprise one or more of the following:
a. communication means;
b. memory means;
c. a GPS device;
d. a loudspeaker;
e. a microphone;
f. an input device;
g. internal communication means for communicating with sensors that are located at remote or not easily accessible locations on the body; and
h. means for waking the patient from an unconscious state.

The system of the invention can be portable and attached to the body of the patient as he carries out his normal daily routine or it can be designed for stationary use at home or in a hospital, clinic, doctor's office, or similar setting.

All the above and other characteristics and advantages of the invention will be further understood through the following illustrative and non-limitative description of preferred embodiments thereof.

### BRIEF DESCRIPTION OF DRAWINGS

FIG. 1 is a flowchart depicting an example of how the abnormal value of a single parameter is used to select the two or more parameters to be used to determine the value of the risk parameter alpha;

FIG. 2 schematically shows two examples that can result in miss alarm based on PTT signal together with pulse rate; and

FIG. 3 is a flow chart showing schematically how the method of the invention is executed, including self-learning.

### DETAILED DESCRIPTION OF PREFERRED EMBODIMENTS

The present invention relates to a method and system for the detection and warning of the presence of pre-fainting and/or other conditions that could be hazardous to a patient with any given type of disease/disorder. The invention accomplishes this purpose by monitoring a wide range of physiological and physical parameters and logically and/or mathematically combining at least two of the monitored parameters, selected according to the patient's known pathological condition, to determine the value of a new parameter called herein risk parameter alpha.

The physiological parameters can be measured by many different means, most of which are well known in the art. For the purposes of the invention the physiological parameters can be measured by means of sensors on devices that are either portable or stationary. The sensors can be components of a device/s that are attached to the patient continuously, only at times of need, or at certain time intervals. The device/s comprising the sensors may be attached to the patient in any appropriate manner so as to measure the necessary physiological parameters, as detailed herein below. Furthermore, the sensors may be connected to the patient either invasively or non-invasively at any appropriate body site. Invasive measurements are performed mainly at home or in hospitals, clinics etc., using stationary systems according to the present invention.

According to a preferred embodiment of the present invention the sensors are components of a portable device attached to the patient at one or more sites, e.g., the wrist, the ankle, the chest, or the patient's breath can be collected using a nasal/oral cannula and End-Tidal Carbon Dioxide (EtCO₂) analyzed with a capnograph.

In addition to physiological parameters physical parameters such as the number of steps taken, steps rate, i.e. number of steps per unit time, and an indication of physical movement of the body as a whole or parts of the body can be included in the function used to determine the instantaneous value of risk parameter alpha at a given moment in time t, which is designated herein as alpha(t) in order to evaluate if changes in the physiological parameters such as heart rate and blood pressure are related to physical activity. Another example of the use of physical parameters is a sensor capable of determining mechanical movement can be used to evaluate the reliability of SPO2 readings since these are affected by movement of the pulse oximeter probe. An example of a sensor that could be used to measure physical parameters relative to the invention is a pedometer, e.g. aGoGYM model TG-224 device.

Once the sensors are attached to the patient, they gather the physiological parameters required for the analysis of the patient's condition. The physiological parameters gathered according to the present invention include, but are not limited to, some or all of the following:
a. heart rate;
b. low frequency modulation of pulse rate (associated with changes in blood pressure and/or breath rates);
c. oxygen saturation;
d. breath rate;
e. heart rhythm, including the detection of atrial and ventricular arrhythmias, any premature beats, or nodal rhythm;
f. body temperature;
g. blood sugar;
h. quantities of any electrolyte, including, but not limited to, sodium, potassium, magnesium, and phosphorus;
i. blood acid base balance as measured by PH;
j. PCO₂ levels (wherein PCO₂ is the partial pressure of carbon dioxide);
k. blood pressure;
l. blood flow;
m. tissue conductivity;
n. SPO₂ (wherein SPO₂ is the saturation of peripheral oxygen);
o. degree of sweating;
p. blood flow in small vessels;
q. Pulse Transit Time (PTT) which, as known to those familiar with the art, may be measured according to pulses at two different locations on the body or according to the time difference between the R-wave and the blood volume pulse;
r. ECG (1, 3 or 12 leads);
s. impedance plethysmography;
t. acoustic breath detection;
u. drug levels ((including for example Digoxin, anti epileptic drugs, and anti arrhythmic drugs);
v. acid-base balance in the serum; and
w. muscle tone measurement.

Once the appropriate parameters have been collected they are analyzed according to the method of the present invention, and compared to normal values by a processing unit in the system of the present invention. In principle, the collected parameters can be analyzed automatically by the system of the invention by any existing method known in the art capable of analyzing such data, or by trained personnel who receive all measurements in real-time via a communications device incorporated into the system.

The average values of the measured parameters are determined for the patient himself from his history or by statistical methods from groups of patients having similar characteristics and health histories. These average values are used to determine the value of a new parameter called herein risk parameter alpha. Risk parameter alpha can be determined from a single parameter (see example 7 herein below); however, according to the preferred embodiment of the present invention at least two of the monitored parameters are logically and/or mathematically combined in a function to determine the value of risk parameter alpha. The parameters selected to be included in the function used to determine alpha are those that have been found to be most clearly related to pre-fainting conditions for a given pathological condition or combination of conditions. Therefore, the function used to determine alpha might be different for each patient or groups of patients. The combination of at least two parameters produces a high level of accuracy in the results, ensuring that the patients are promptly treated when any problems arise, and furthermore, ensuring that the number of false alarms be kept at a minimum.

The method and system are designed to give both increased selectivity and increased specificity, thereby increasing reliability, by deriving alpha from at least two parameters. The higher accuracy in alarms using two parameters results from: (i) better understanding of physiological status for example, by correlating changes in PTT and heart rate or in another example correlating between physical activity as derived from the step counter and changes in PTT or; (ii) the possibility of addressing measurement challenges/limitations, for example by ignoring changes in SPO2 during movement of the patient or in another example ignoring the PTT parameter when the pulse rate reading is not reasonable.

An outline of the way in which the invention accomplishes its purpose follows. The steps of the outline will be described in more detail with respect to the figures hereinbelow.

The main steps in the method of the invention are:
a. monitoring a wide range of physiological and physical parameters;
b. logically and/or mathematically combining at least two of the monitored parameters to form a function used to determine the value of a new parameter called herein risk parameter alpha, wherein the parameters that appear in the function are selected according to the patient's known pathological condition;
c. determining an initial threshold value (alpha) based on known normal values of the monitored parameters as determined by statistical studies;
d. using the function to determine the current value of alpha, defined as alpha(t),
e. comparing alpha(t) to the threshold value of alpha;
f. emitting a warning signal if the comparison shows that there exists danger of the onset of a pre-fainting and/or other medically hazardous condition conditions;
g. continually determining and, if relevant, updating the initial value of alpha according to the history of the patient; and
h. continually determining and, if relevant, updating the terms, i.e. weighting factors, and parameters that comprise the function used to determine alpha(t) according to the history of the patient.

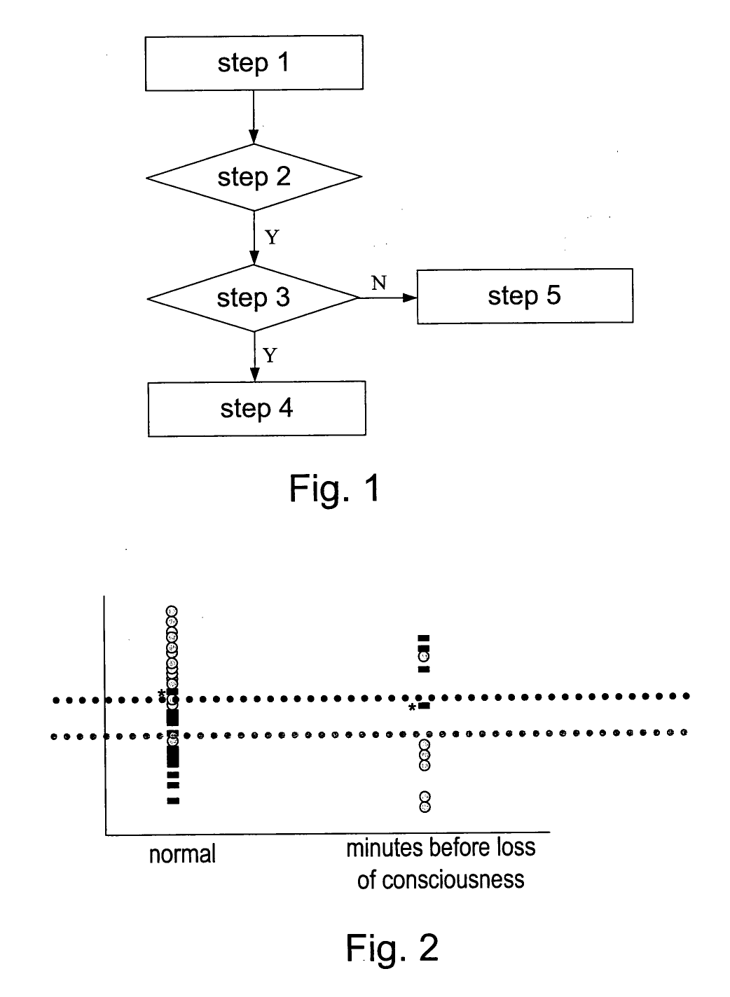

FIG. 1 is a flowchart depicting an example of how the abnormal value of a single parameter is used to select the two or more parameters to be used to determine the value of the risk parameter alpha. In step 1 of FIG. 1 the pulse is measured. The measurements can be made either continuously, on demand, or at specified time intervals according to a decision made automatically in the processing unit of the system of the invention or manually by the subject or his doctor. In step 2 the measured pulse rate is compared with a range of normal values determined for the subject taking into account various factors such as gender, age, physical condition, etc. If it is determined that the pulse rate is abnormal, then in step 3 a determination is made if the pulse rate is too low. If the pulse rate is too low there exists the risk of bradycardia and the system is instructed in step 4 to initiate measurements of the SPO₂ and tissue conductivity and, according to the results, also the blood pressure. If the abnormal pulse rate is not too low, i.e. it is too high, there is a risk of tachycardia and the system is instructed in step 5 to initiate measurements of blood pressure and 1-lead-ECG.

FIG. 2 schematically shows how the use of two parameters to determine alpha(t) can, on the one hand, prevent a false alarm that would be issued based on the use of only one parameter and, on the other hand, result in the issuance of an alarm that would be missed based on the use of only one parameter.

In the figure the rectangles represent the data for the pulse/heart rate, the circles represent the PPT, and the upper and lower dotted horizontal lines represent thresholds for the pulse rate and PPT respectively. The value of the parameters is measured along the vertical axis and the data points can represent either a single measurement or the average of a number of measurements. The left hand column shows the normal values for the patient and the right hand column shows the values of the parameters measured a few minutes before the same patient lost consciousness either naturally or induced under controlled conditions. From data such as that shown in FIG. 2, it can be seen that if an instantaneous value of the first of the parameters seems abnormal but the value of the second parameter paired with it is clearly in the normal range, then a false alarm (that would be issued based on the first parameter alone) can be avoided. On the other hand, if the value of the first parameter seems normal, even if very close to the threshold while the value of the second parameter is clearly abnormal an alarm that would be missed based on the first parameter alone will be issued.

The collected data for each of the parameters at a given time are used to calculate the instantaneous value of the risk parameter alpha(t). Alpha(t) is then compared to the normal value for alpha, which is determined from the normal values for each parameter. The normal values of the parameters are known from previously gathered statistical population based data and are preferably tailored as closely as possible to the health and personal profile of the subject. In another embodiment the normal value is not determined for each specific parameter but for the combination of parameters used to calculate alpha (t), i.e. normal values can be based on the expected average and fluctuations of alpha(t) determined from the characteristics of a specific patient/subject.

The preferred embodiment of the present invention has self-learning abilities, which enable the function used to determine alpha(t) and the value of alpha to be updated as new information becomes available. In particular alpha is updated in accordance with the values of the physiological parameters of the subject that are measured before and during a fainting episode. In this way the ability of the system to accurately predict a pre-fainting condition for the subject is increased with time. Self learning can involve adjusting the value of alpha if an event is missed, e.g. if alpha(t) remains below the “normal value” of alpha as determined for the general population for a period of 24 hours before a pre-fainting episode occurs. In this case, the value of alpha is adjusted upward. Alternatively, self learning occurs when false alarms occur, e.g. an alpha(t), which should have been accompanied by a pre-fainting episode, is determined from measured parameters; however such an episode did not occur. In this case the value of alpha will be adjusted downward. Self learning can also include modifying the function used to generate alpha (t) by adjusting the weighting factors which determine the relative contribution of each of the parameters, by adding new parameters, or by selecting a different function used to determine alpha(t).

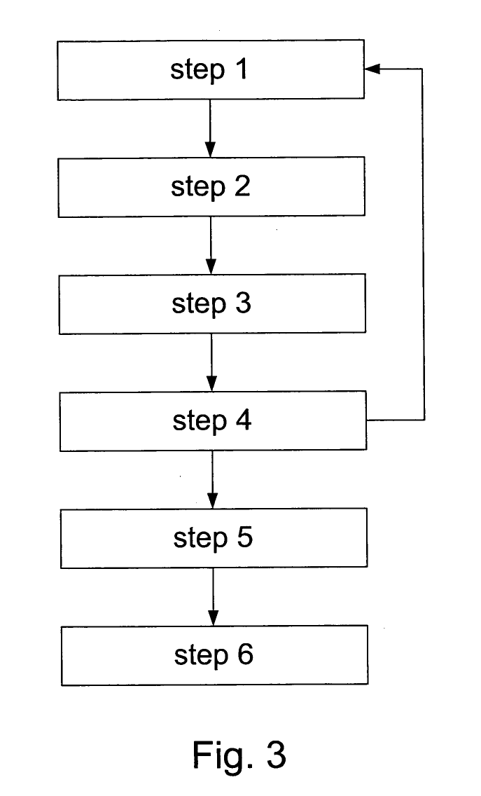

FIG. 3 is a flow chart showing schematically how the method of the invention is executed, including self-learning. In step 1 the function used to calculate alpha(t) and the initial threshold value of risk parameter alpha are determined by determining the individualized normal values for each of the tested physiological parameters based on the subject's medical history, basic disorders, medications, etc. In step 2, measurements are carried out to determine values of alpha(t). In step 3 the patient experiences a pre-syncope, either naturally or intentionally induced by a maneuver performed by medical personnel. In step 4, the values of the parameters measured in step 3 are used to determine a new function and/or threshold value of alpha that is returned to step 1. At the same time as the self-learning is taking place in step 4, alpha(t) is compared with the current value of alpha. In step 5, it is determined if the threshold has been crossed. If it has, then in step 6 a signal is sent that alerts the subject or other persons, activates a medical device, or causes the system of the invention to begin measuring additional parameters in order to provide more detailed information.

If the alpha(t) for a patient deviates from the updated value of alpha derived for him in such a manner that may point to a pre-fainting condition, then an warning is issued and an appropriate party is notified. The appropriate party notified of any problems may be the patient himself or a friend, relative, or care-giver responsible for that patient.

It is to be noted that herein words such as “alarm” and “warning” are used in a generic sense to refer to a signal or notification sent from the processing system to the patient or others regarding the condition of the patient, i.e. if his condition is normal or if he is entering into a pre-fainting or otherwise hazardous condition. It should be noted that the alarm is not necessarily a simple “yes” or “no”, but in preferred embodiments the system of the invention presents the probability of the condition. The words “alarm” or “warning” can also refer to signals sent by the processing units to activate devices that act to alleviate the condition, e.g. an insulin pump. “Alarms” can have any form and be issued be any method known in the art, for example: a silent alarm could be a notice on a display screen; a tactile alarm could be an electric shock, and an audible alarm could be issued by the processing system via an internal loudspeaker.

In order for the notification to be able to reach remote parties, the system of the present invention comprises communications means, which are preferably wireless two-way communication means. As a result of this capability the system allows remote parties, such as personnel at an emergency service center, to receive data in real-time and to respond for example, by sending voice messages to the patient or commands to the system regarding additional parameters that should be monitored. The communication means may operate according to any known technology, e.g. cellular phone or Bluetooth technology, and may be equipped to send messages of any suitable type, e.g. voice, email, or SMS.

In one embodiment of the present invention, if the notified party is the patient and he does not react by turning off the alarm, the system automatically alarms a further party who can come to the aid of the patient. This is expected to be especially important when the patient is incapable of reacting due to his medical condition. In this embodiment the further party may be an emergency service, which is contacted by the system of the present invention and in response automatically sends an ambulance to the patient's location. In this embodiment, a GPS device can be provided to enable the patient to be easily located if necessary.

In another embodiment of the present invention the notification is sent, either additionally or exclusively, to a medical device attached to the patient, e.g. an insulin pump or pacemaker, thereby allowing that device to automatically treat the patient selectively according to his present condition.

As said herein above, the system of the invention is preferably portable and attached to the body of the subject as he carries out his normal daily routine. In some embodiments it is designed for stationary use at home or in a hospital, clinic, doctor's office, or similar setting. In either case the main components of the system are the same. They comprise a processor; sensors to measure the appropriate physiological and physical parameters; a power supply, e.g. rechargeable batteries for portable systems and mains power for stationary systems; and optionally, communication means, which for portable systems preferably allow two-way communication. The system should preferably comprise memory means to establish a historical record of the readings of the various sensors, values of alpha(t), a record of the functions used to determine alpha(t), updated values of alpha, and any relevant information manually entered by the patient or others. The system can also comprise other devices such as a GPS device, loudspeaker, microphone, and input device such as a keypad. Embodiments of the system of the invention comprise internal communication means for communicating with sensors that are located at remote or not easily accessible locations on the body, for example implanted or swallowed bio-chips, which may aid both in diagnostics and the treatment of the patient. In a specific embodiment of the present invention the system comprises means for waking the patient from unconsciousness, e.g. low power high voltage signals.

The systems of the invention will be designed to carry a wide range of sensors. The portable systems will comprise a minimal number of sensors selected to provide the data necessary to determine the risk parameter alpha tailored according to the specific profile of the subject. The stationary systems will be equiped with sensors capable of measuring a much wider range or parameters and will be designed for use with a general population of subjects that can suffer from a wide range of medical conditions.

A few non-limiting examples of functions used to determine the risk for a specific patient at a specific time, i.e. alpha(t) follow; wherein, the same functions can be used to determine the value of threshold (alpha), which provides the most reliable alarm. It is to be noted that, although for clarity purposes, specific approaches are described in specific examples it is emphasized that the examples are given only to illustrate the method of forming the function for a particular patient and preferred embodiments of the invention are based on combinations of several different approaches of the types illustrated herein.

### Example 1

This example illustrates how a function that can be used to determine the value of risk parameter alpha(t) can be generated from a number of physiological parameters at time t for a specific subject, who is known or suspected to be suffering from a cardiovascular condition:

alpha(t)=a*(pulse rate(t)−average pulse rate)/STD of pulse rate+b*(PTT(t)−average PTT)/STD of PTT+c*ABSOLUTE VALUE(breath−rate(t)−average breath rate)/STD of breath rate+d*(body temp(t)−37)

In this and the following examples:
a, b, c, d, etc. are constant weighting factors that are determined empirically from a representative population by known methodologies such as linear regression or logistic regression;
the STD values of the parameters are taken from statistical studies of groups of patients having the same pathological condition;
the initial average values are derived from the patient's parameters in relevant conditions; and
If |alpha(t)|>X, where X is a predetermined constant, and alpha(t) has a predetermined sign, then the system transmits an alarm or initiates the testing of other parameters.

Learning can be implemented by at least one of the following methods; (i) The average and or STD values are originally statistical values derived from a general population. As time passes and data connected to the subject/patient is accumulated the statistical values are replaced with those specific to the subject. (ii) The constants, i.e. weighting factors, a, b, c, and d are adjusted to provide the best discrimination between normal vs. pre-faint conditions on the same patient. (iii) the threshold values to determine when an alarm is needed might be adjusted to improve reliability.

### Example 2

This example how a function that can be used to determine risk parameter alpha(t) can be generated from a number of physiological parameters for a member of an elderly population with cryptogenic history of pre-fainting or patients with suspected neurological disorders for a specific subject at time t, wherein the natural logarithm (Ln) of combinations of the parameters or combinations of the parameters raised to a power >1, are used:

alpha(t)=a*[(pulse rate(t)−average pulse rate)/STD of pulse rate]ⁿ+b*[(PTT(t)−average PTT)/STD of PTT]^{m}+c*ABSOLUTE VALUE [(breath-rate(t)−average breath rate)/STD of breath rate]ₚ+d*(body temp(t)−37)^{q}+e*Ln(Tissue conductivity−average tissue conductivity)

### Examples 3

The following examples illustrate how a function that can be used to determine risk parameter alpha(t) can be generated for patients with abnormal blood pressure from a number of physiological parameters for a specific subject at time t and wherein interaction between parameters is introduced.

### Example 3a

The following is an example wherein some of the parameters interact with each other and the deviation from normal is exponential:

alpha(t)=a*exp{b*[(pulse rate(t)−average pulse rate)/STD of pulse rate]}+c*exp{d*[PTT(t)−average PTT)/STD of PTT]}+f*exp{g*[(Pulse rate−Average Pulse rate)/(Tissue conductivity-average tissue conductivity)]}+h*(breath rate(t)−average breath rate)/STD of breath rate+i*(body temp(t)−37)

Note that the factors can be either positive or negative according to the results from the regression; therefore the relevant signs have to be chosen.

### Example 3b

Additional interactions/inter-relation between parameters can be implemented. For example, the contribution of a specific parameter, such as pulse rate, can depend on the value of another parameter such as steps rate in such a way that if movement of the patient above a given speed is detected, then the value of weighting factor a is set to zero in order to avoid non-relevant information which is associated with the motion. The following is an example of a function used to determine alpha(t) in accordance with these principles:

alpha(t)=a*exp{b*[(pulse rate(t)−average pulse rate)/STD of pulse rate]}+c*exp{d*[(PTT(t)−average PTT)/STD of PTT]}+f*exp{g*[(Pulse rate−Average Pulse rate)/(Tissue conductivity−average tissue conductivity)]}+h*(breath rate(t)−average breath rate)/STD of breath rate+i*(body temp(t)−37)

Wherein, a=0 when >3 steps per minute are detected.

### Example 3c

A more advanced interactions/inter-relation between parameters can be implemented. For example one in which the contribution of a specific parameter, such as pulse rate, can depend on the value of another parameter such as steps rate; wherein the pulse rate is normalized by the steps rate in a manner such that the expected increase in pulse rate due to movement doesn't lead to a false alarm. For this example:

alpha(t)=a*exp{[(pulse rate(t)/(steps rate−b)^{c}−average pulse rate at rest]}+d*exp{*[(PTT(t)−average PTT)/STD of PTT]}+f*exp{g*[(Pulse rate−Average Pulse rate)/(Tissue conductivity−average tissue conductivity)]}+h*(breath rate(t)−average breath rate)/STD of breath rate+i*(body temp(t)−37)

### Examples 4

The following examples illustrate how a function that can be used to determine the value of risk parameter alpha(t) can be generated from a number of physiological parameters for a specific subject at time t, wherein some of the parameters are structured/modeled in a manner that generate risk for a pathology/acute conditions, as conventionally used in logistic regression analysis. The parameter/s can be structured to be linear, multivariate, exponential and more. (The values used to derive the model can be the patient's parameters in normal and acute fainting conditions and/or statistical parameters from a relevant population).

### Example 4a

In this example the pulse rate is structured in a term having the form of Exp(a+b*parameter)/[1+Exp(a+b*parameter)] and other parameters are structured in terms having a different format.

alpha(t)={A*[exp((a*pulse rate(t)+b))/[1+exp(a*pulse rate(t)+b)]+B*exp[(PTT(t)−average PTT)/STD of PTT]+c*exp[(Pulse rate−Average Pulse rate)/(Tissue conductivity−average tissue conductivity)]+[breath rate(t)−average breath rate/2STD of breath rate+d[(body temp−37)/2]}

In determining if the system should transmit an alarm or initiation the testing of other parameters, it is important to take into account that in logistic regression the values of alpha(t) will be from 0 to 1 and different in others models therefore, factoring is require.

### Example 4b

In this example the pulse rate and PPT are structured in one term and the other parameters in structured in terms having a different format.

alpha(t)={A*[exp((a*pulse rate(t)+b*(PTT(t)+c))/*[1+exp(a*pulse rate(t)+b*PTT(t)+c)+C*[breath−rate(t)−average breath rate/2STD of breath rate]+d[(body temp−37)/2]}

### Example 4c

In this example only logistic regression is used and the probability of a pre-fainting condition is derived from a combination of several parameters chosen such that they interact with each other.

alpha(t)={exp(a*pulse rate(t)+b*PTT(t)+c*[(bodytemp(t)₁-body temp(t)₂)/(bodytemp(t)₁−37)/]+d/[1+(exp(a*pulse rate(t)+b*PTT(t)+c*[(bodytemp(t)₁−body temp(t)₂)/(bodytemp(t)₁−37)]}

Wherein bodytemp(t)₁ is the body temperature at position 1 and bodytemp(t)₂ is the body temperature at position 2, both at time t. In this format the probability of problem/acute conditions, i.e. the value of alpha(t), is derived automatically from 0 to 1.

### Example 5

In parameters for a specific subject at time t, wherein the temp is derived from two different locations in the body.

alpha(t)=a*[(pulse rate(t)−average pulse rate)/STD of pulse rate]ⁿ+b*[(PTT(t)−average PTT)/STD of PTT]^{m}+c*[breath−rate(t)−average breath rate/2STD of breath rate]^{P}+d*[(body temp in site 1−body temp in site 2)/2]^{q}

### Example 6

In this example a function used to determine risk parameter alpha(t) is generated from number of physiological parameters for a specific subject at time t, wherein the rate of change of a parameter in the last m minutes is calculated.

Parameter alfa(t)=a*[(pulse rate(t)−pulse rate(t−m))/STD of pulse rate]ⁿ+b*[(PTT(t)−PTT(t−m))/STD of PTT]^{m}+[breath-rate(t)−average breath rate/2STD of breath rate]^{P}+d[(body temp in site 1−body temp in site 2)/2]^{q}

### Examples 7

As said previously, it is preferred to use measurements of at least two parameters to determine alpha(t) because of the advantages derived from this as discussed herein; however, embodiments of the invention may comprise an initial step of using the measurement of a single parameter in order to give a first indication of when an abnormal condition is about to take place. In this case the measured value of alpha(t) is compared to a standard value. In some circumstances, for example if the deviation of the measured value of alpha(t) from the normal is above a predetermined value, than a warning signal can be sent based on the measurement f one parameter only. Normally, however, deviation of alpha(t) from the normal initiates measurement of predetermined additional parameters to determine a more reliable alpha(t) as illustrated in the above examples. The decision concerning the additional parameters to be measured may be automatically performed by the system of the present invention, or by any other appropriate means, including instructions sent to the device of the invention by medical staff receiving the result/s of the measurement/s from the system in real-time.

It is to be noted that a similar procedure can be used when the initial measurements are for more than one parameter. For example, deviation of alpha(t) calculated on the basis of input from two sensors from the normal can initiate measurement of one or more predetermined additional parameters in order to calculate a new alpha.

Operating the system in this manner is advantageous, assuming relevant information about the patient's medical condition can be extracted from a single parameter, since, for example, it allows simpler measurement and analysis of the data and considerable energy savings since only one parameter need be measured until it is determined that additional information is needed, at which point additional sensors are activated.

### Example 7a This example shows a function used to determine the risk parameter alpha(t) by using measurement of pulse rate wherein the value of the pulse rate at time (t) as well as the trend, i.e. the change in value, in the last x minutes are measured.

alpha(t)=a*[(pulse rate(t)−average pulse rate)/STD of pulse rate]ⁿ+b*[(pulse rate(t)−pulse rate(t−X)−)/c*STD of pulse rate]^{m}

### Example 7b

This example shows a function used to determine the risk parameter alpha(t) by measurement of pulse transit time (PTT) wherein the value, trend in the last Y minutes, and fluctuations, i.e. physiological noise in the last Z minutes of the PTT are measured and used.

alpha(t)=a*[PTT(t)−average PTT)/STD of PTT]ⁿ+b*[(PTT(t)−pulse rate(t−Y)−)/c*STD of PTT]^{m}+d*STD(PTT(t to t−z))

### Example 8

As it is known in the art that a specific sensor can provide information that relates to several parameters. For example, from the pulse rate measurement parameters which are associated with Breath Rates (BRₚᵤₗₛₑ) and changes in Blood Pressure (BPₚᵤₗₛₑ) based on low frequency modulations, noise etc, can be derived. The following example includes such parameters together with PTT signal and SPO2 measurement and Breath Rate derived from acoustic measurement (BR_{acoustic}) in a manner that together provides a more reliable alarm than single parameters.

alpha(t)={a*[(SPO2(t−averageSPO2)/STD of SPO2]ⁿ+b*[(pulse rate(t)/average of pulse rate]^{m}+c*[(PTT(t)−average PTT)/(PTT(t)−d*PBₚᵤₗₛₑ+e)+f*[BRₚᵤₗₛₑ(t)−average BRₚᵤₗₛₑ(t)/(BRₚᵤₗₛₑ(t)−BR_{acoustic}(t)+g)]}

Wherein the factors a-g, m, and n can be configured in the function and their values set initially according to the characteristics of a general patient or group of patients and adjusted as part of the learning process for a specific subject.

Although embodiments of the invention have been described by way of illustration, it will be understood that the invention may be carried out with many variations, modifications, and adaptations, without exceeding the scope of the claims.

1. A method for the detection, qualitative evaluation, and warning of the presence of pre-fainting and other conditions that are hazardous to the health of a patient having one or more types of disease/disorder; said method comprising the following steps:
a. monitoring at least one physiological parameter selected according to the patient's known pathological condition;
b. determining the instantaneous value of the risk parameter (alpha(t));
c. assigning to alpha(t) at least one threshold value (alpha) whose value is determined based on known normal values as determined by statistical studies;
d. comparing the value of alpha(t) to the current value of alpha;
e. emitting a warning signal if the comparison shows that the value of alpha(t) is different from the value of alpha by an amount that exceeds a value predetermined for said patient;
f. using said instantaneous monitored values of said parameter to update alpha(t); and
g. repeating steps d to f.

2. The method according to claim 1, comprising the additional step of re-determining and if relevant updating said current value of alpha according to the history of the patient between steps e and f.

3. The method according to claim 1, wherein emitting a warning signal comprises presenting the probability that a pre-fainting or other condition that is hazardous to the health of the patient is occurring or will occur.

4. The method according to claim 1, wherein self-learning techniques are used to assist in continually updating the value of alpha.

5. The method according to claim 1, wherein self-learning techniques are used to assist in continually updating a function used to determine the value of alpha(t).

6. The method according to claim 1, wherein:
a. at least one additional physiological or physical parameter, which is selected according to the patient's known pathological condition, is monitored;
b. the instantaneous value of the risk parameter (alpha(t)) is determined from a function that combines the measured values of said one selected physiological parameter and of said at least one additional parameter; and
c. the threshold value (alpha) is determined by statistical studies.

7. The method of claim 6, wherein combination of the measured values of the parameters is done mathematically.

8. The method of claim 6, wherein combination of measured values of the parameters is done logically.

9. The method of claim 6, wherein the threshold value (alpha) is determined by combining the known normal values of the selected parameter and the known normal values the at least one additional parameter.

10. The method of claim 6, wherein the threshold value (alpha) is determined by using normal values of the combination of the selected parameter and the at least one additional parameter.

11. The method according to claim 6, wherein self-learning techniques are used to assist in continually updating one or both of the terms and parameters that comprise the function used to determine the value of alpha(t) and the threshold value (alpha).

12. The method according to claim 1, wherein, instead of emitting a warning signal, a new parameter is selected and the steps of the method are carried out using said new parameter.

13. The method according to claim 6, wherein, instead of emitting a warning signal, a new set of parameters comprising additional or different parameters is selected and the steps of the method are carried out using said new set of parameters.

14. The method according to claim 1, wherein the physiological parameters monitored are selected from the list comprising:
a. heart rate;
b. low frequency modulation of pulse;
c. oxygen saturation;
d. breath rate;
e. heart rhythm, including the detection of atrial and ventricular arrhythmias, any premature beats, or nodal rhythm;
f. body temperature;
g. blood sugar;
h. quantities of any electrolyte;
i. blood acid base balance;
j. PCO₂ levels;
k. blood pressure;
l. blood flow;
m. tissue conductivity;
n. SPO₂;
o. degree of sweating;
p. blood flow in small vessels;
q. Pulse Transit Time;
r. ECG;
s. impedance plethysmography;
t. acoustic breath detection;
u. drug levels;
v. acid-base balance in the serum; and
w. EtCO₂.

15. A method according to claim 6, wherein the physical parameters are selected from the list comprising:
a. number of steps taken;
b. steps rate;
c. an indication of physical movement of the body as a whole; and
d. an indication of physical movement of parts of the body.

16. A system for carrying out the method of claim 1, said system comprising:
a. a processor;
b. at least one sensor to measure the appropriate physiological and physical parameters; and
c. a power supply.

17. A system according to claim 16 additionally comprising one or more of the following:
a. communication means;
b. memory means;
c. a GPS device;
d. a loudspeaker;
e. a microphone;
f. an input device;
g. internal communication means for communicating with sensors that are located at remote or not easily accessible locations on the body; and
h. means for waking the patient from an unconscious state.

18. A system according to claim 16, wherein said system is portable and attached to the body of the patient as he carries out his normal daily routine.

19. A system according to claim 16, wherein said system is designed for stationary use at home or in a hospital, clinic, doctor's office, or similar setting.
